SEQUENCE LISTING (8 sequences)

SEQ ID NO: 1 - protein, 128 aa, Homo sapiens
QVQLVESGGGVVQPGRSLRLSCAASGFTFSSYGMHWVRQAPGKGLEWVAVMWYDGSNKDYVDSVKGRFTISRDNSKNTLYLQMNRLRAEDTAVYYCAREKDHYDILTGYNYYYGLDVWGQGTTVTVSS

SEQ ID NO: 2 - protein, 107 aa, Homo sapiens
DIQMTQSPSSLSASVGDRVTITCRASQGIRNDLGWYQQKPGKAPKRLIYAASSLQSGVPSRFSGSGSGTEFTLTISSVQPEDFVTYYCLQHNSNPLTFGGGTKVEIK

SEQ ID NO: 3 - protein, 5 aa, Homo sapiens
SYGMH

SEQ ID NO: 4 - protein, 17 aa, Homo sapiens
VMWYDGSNKDYVDSVKG

SEQ ID NO: 5 - protein, 19 aa, Homo sapiens
EKDHYDILTGYNYYYGLDV

SEQ ID NO: 6 - protein, 11 aa, Homo sapiens
RASQGIRNDLG

SEQ ID NO: 7 - protein, 7 aa, Homo sapiens
AASSLQS

SEQ ID NO: 8 - protein, 9 aa, Homo sapiens
LQHNSNPLT

(19) United States
(12) Patent Application Publication
(10) Pub. No.: US 2019/0002577 A1
(43) Pub. Date: Jan. 3, 2019

(54) Methods Of Treating Type 2 Diabetes Mellitus Using Glucagon Receptor Antagonistic Antibodies

(71) Applicant: REMD Biotherapeutics, Inc., Camarillo, CA (US)

(72) Inventors: Jim Shi, Thousand Oaks, CA (US); Hai Yan, Thousand Oaks, CA (US)

(73) Assignee: REMD Biotherapeutics, Inc., Camarillo, CA (US)

(21) Appl. No.: 16/068,218

(22) PCT Filed: Jan. 4, 2017
(86) PCT No.: PCT/US17/12220

§ 371 Date: Jul. 5, 2018

Related U.S. Application Data
(60) Provisional application No. 62/274,662, filed on Jan. 4, 2016.

(51) Int. Cl. C07K 16/28; A61P 3/10

(52) U.S. Cl. CPC C07K 16/2869; A61K 2039/505; C07K 2317/76; A61P 3/10

(57) ABSTRACT

The present invention relates to methods of treating Type 2 diabetes (T2D), comprising administering to a patient an effective amount of an isolated antagonistic antigen binding protein that specifically binds to the human glucagon receptor, either as monotherapy, or in combination with one or more antidiabetic medications.

### RELATED PATENT APPLICATIONS

This application claims benefit of U.S. Provisional Application No. 62/274,662, filed on Jan. 4, 2106, incorporated by reference in its entirety.

### TECHNICAL FIELD

Type 2 Diabetes Mellitus (T2D) (formerly noninsulin-dependent diabetes mellitus (NIDDM) or adult-onset diabetes) is a metabolic disorder that is characterized by hyperglycemia (high blood sugar) in the context of insulin resistance and relative lack of insulin. Type 2 diabetes is an established and growing health concern worldwide. Chronic hyperglycemia is associated with damage and dysfunction of various organs, including, but not limited to the eye, kidney, nerves, and heart. As of 2010, there were approximately 285 million people diagnosed with the disease compared to around 30 million in 1985, and Type 2 diabetes makes up about 90% of cases of diabetes. This is equivalent to about 6% of the world's adult population. Obesity is thought to be the primary cause of Type 2 diabetes in people who are genetically predisposed to the disease (although this is not the case in people of East-Asian ancestry).

Traditional first-line treatment for managing T2D starts with healthy lifestyle choices, e.g., diet, exercise and weight control, but often includes medications, e.g., metformin or insulin, to achieve target blood sugar (glucose) levels. Of adults diagnosed with diabetes (Type 1 or Type 2), 16% take no medication; 58% take only oral medication; 14% take insulin and oral medication; and 12% take insulin only, according to the U.S. Center for Disease Control. Although long-term complications of diabetes develop gradually, they can eventually be disabling or even life-threatening. Some of the potential complications of diabetes include: heart and blood vessel disease; nerve damage (neuropathy); kidney damage (nephropathy); eye damage; foot damage; hearing impairment; skin conditions; and Alzheimer's disease.

Several classes of T2D medicines exist. Each works in different ways to lower blood sugar. A drug may work by: stimulating the pancreas to produce and release more insulin; inhibiting the production and release of glucose from the liver; blocking the action of stomach enzymes that break down carbohydrates; improving the sensitivity of cells to insulin; inhibiting the reabsorption of glucose in the kidneys; or slowing how quickly food moves through the stomach. Oral medications include biguanides (e.g., metformin), meglitinides, sulfonylureas, dipeptidyl-peptidase 4 (dpp-4) inhibitors, thiazolidinediones, alpha-glucosidase inhibitors, sodium-glucose transporter 2 (SGLT2) inhibitors, and bile acid sequestrants. Injectable medications include amylin mimetics and incretin mimetics. While demonstrating various degrees of effectiveness, more than half of patients with type 2 diabetes today are not meeting their HbA1c treatment goals using the currently available therapies.

Because Type 2 diabetes is often associated with a relative excess of glucagon, which stimulates hepatic glucose production, pharmacologic agents that safely and effectively inhibit glucagon action represent a promising approach to the treatment of the disease; Basu et al., Am J Physiol Endocrinol Metab, 287(1):55-62, 2004; Bock et al., Diabetes, 55(12):3536-3549, 2006. Several approaches to inhibiting glucagon signaling have been explored to date. One approach, using small molecule glucagon receptor (GCGR) antagonist has various programs in preclinical and early clinical studies; see, e.g., Kazda et al., (Abstract), 74th Scientific Sessions, Am Diabetes Assoc., 2014. Another approach, using monoclonal antibodies to inhibit glucagon directly, failed due to the induction of compensatory glucagon secretion.

Gestational diabetes mellitus (“GDM”) is another disorder associated with elevated circulating plasma glucose. GDM has many features in common with type 2 diabetes. The endocrine (impaired insulin secretion) and metabolic (insulin resistance) abnormalities that characterize both forms of diabetes are similar. In general, pregnancy is characterized by increases in both insulin resistance and insulin secretion. Women with GDM fail to respond with increased insulin to the decrease in insulin sensitivity. GDM pregnancies are at an increased risk for fetal macrosomia and neonatal morbidities including neural tube defects, hypoglycemia, hypocalcemiea, hypomagnsemia, polycythemia and hyperbilirubinemia and subsequent childhood and adolescent obesity (Grigorakis et al., Gynecol Obstet Invest, 49:106-109, 2000).

In both acute and chronic nonclinical settings, various animal model studies in mouse and monkeys have shown that an antagonistic human monoclonal antibody capable of blocking the glucagon receptor has profound glucose-lowering effects without inducing hypoglycemia (Yan, et al., J Pharmacol Exp Ther., 329(1):102-11, 2009). Further, it has been demonstrated that targeting glucagon production or function using isolated antagonistic antigen binding proteins that specifically bind to and antagonize the human glucagon receptor (GCGR) are capable of controlling and lowering blood glucose, and improving glucose tolerance, in Type 2 diabetes models (see, e.g., U.S. Pat. No. 7,947,809 (Yan, et al)). The ability of such antagonistic antigen binding proteins to effectively treat patients who have Type 2 diabetes or who are at risk of developing Type 2 diabetes has not yet been fully evaluated.

### DISCLOSURE OF THE INVENTION

The present disclosure is based, in part, on the inventors' unique insight that an isolated antagonistic antigen binding protein that specifically binds to the human glucagon receptor (e.g., the fully human IgG2 monoclonal antibody referred to herein as “REMD-477”) will be efficacious, safe and well tolerated when administered as a single subcutaneous dose followed by repeated subcutaneous doses in Type 2 diabetes patients. It is envisioned that at least a 15% reduction in fasting blood glucose, as well as at least a 15% reduction in glucose area under the curve (AUC) as determined by mixed meal tolerance test (MMTT) will be observed in patients with T2D after single and/or repeated doses of REMD-477. It is also envisioned that a robust lowering of hemoglobin A1c (HbA1c) levels and increases in serum glucagon and serum GLP-1 levels will be observed in patients with T2D after single and/or repeated doses of REMD-477.

Thus, in one aspect, the present disclosure relates to methods for treating a patient diagnosed with Type 2 diabetes (T2D) comprising administering to the patient a therapeutically effective amount of an isolated antagonistic antigen binding protein that specifically binds to the human glucagon receptor.

In various embodiments, the patients have been diagnosed as having T2D and are either treatment-naïve, or are being controlled with diet and exercise only. In various embodiments, the patient has been diagnosed as having T2D and is being treated with an antidiabetic medication selected from the group consisting of: biguanides (e.g., metformin), sulfonylureas, dipeptidyl- peptidase 4 inhibitors, sodium-glucose co-transporter 2 (SGLT2) inhibitors, α-glucosidase inhibitors, thiazolidinediones, bile acid sequestrants, amylin mimetics and incretin mimetics. In various embodiments, the patient is being treated with stable doses of metformin.

In various embodiments, the T2D is refractory to treatment with an antidiabetic medication selected from the group consisting of: biguanides (e.g., metformin), sulfonylureas, dipeptidyl- peptidase 4 inhibitors, sodium-glucose co-transporter 2 (SGLT2) inhibitors, α-glucosidase inhibitors, thiazolidinediones, bile acid sequestrants, amylin mimetics and incretin mimetics.

In various embodiments, the isolated antagonistic antigen binding protein comprises an antibody selected from a fully human antibody, a humanized antibody, a chimeric antibody, a monoclonal antibody, a polyclonal antibody, a recombinant antibody, an antigen-binding antibody fragment, a Fab, a Fab′, a Fab₂, a Fab′₂, a IgG, a IgM, a IgA, a IgE, a scFv, a dsFv, a dAb, a nanobody, a peptibody, a unibody, or a diabody. In various embodiments, the isolated antagonistic antigen binding protein that specifically binds to the human glucagon receptor is a fully human IgG2 monoclonal antibody which comprises the heavy chain variable region having the amino acid sequence of SEQ ID NO: 1 and a light chain variable region having the amino acid sequence of SEQ ID NO: 2 (the “REMD-477” antibody). In various embodiments, the isolated antagonistic antigen binding protein comprises an antibody comprising a heavy chain comprising Complementarity-Determining Region (CDR)1 having the amino acid sequence of SEQ ID NO: 3, CDR2 having the amino acid sequence of SEQ ID NO: 4, and CDR3 having the amino acid sequence of SEQ ID NO: 5; and a light chain comprising CDR1 having the amino acid sequence of SEQ ID NO: 6, CDR2 having the amino acid sequence of SEQ ID NO: 7, and CDR3 having the amino acid sequence of SEQ ID NO: 8.

In various embodiments, REMD-477 is administered to the patient at a dosage (e.g., at a weekly dosage) included in any of the following ranges: about 0.1 to about 0.2 mg/kg, about 0.2 to about 0.4 mg/kg, about 0.4 to about 0.6 mg/kg, about 0.6 to about 0.8 mg/kg, about 0.8 to about 1.0 mg/kg, about 1.0 to about 1.2 mg/kg, about 1.2 to about 1.4 mg/kg, about 1.4 to about 1.6 mg/kg, about 1.6 to about 1.8 mg/kg, about 1.8 to about 2.0 mg/kg, about 2.0 to about 2.2 mg/kg, about 2.2 to about 2.4 mg/kg, about 2.4 to about 2.6 mg/kg, about 2.6 to about 2.8 mg/kg, and about 2.8 to about 3.0 mg/kg. In various embodiments, REMD-477 is administered to the patient at a weekly dosage selected from the group consisting of .1 mg/kg, .2 mg/kg, .3 mg/kg, .4 mg/kg, .5 mg/kg, .6 mg/kg, .7 mg/kg, .8 mg/kg, .9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, and 3.0 mg/kg.

In various embodiments, REMD-477 is administered to the patient at a dosage (e.g., at a weekly dosage) included in any of the following ranges: about 5 to about 15 mg, about 15 to about 30 mg, about 30 to about 45 mg, about 45 to about 60 mg, about 60 to about 75 mg, about 75 to about 90 mg, about 90 to about 105 mg, about 105 to about 120 mg, about 120 to about 135 mg, about 135 to about 150 mg, about 150 to about 165 mg, about 165 to about 180 mg, about 180 to about 195 mg, and about 195 to about 210 mg. In various embodiments, REMD-477 is administered to the patient at a weekly dosage selected from the group consisting of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 200 mg, 205 mg, and 210 mg

In another aspect, the present disclosure comprises a method for reducing, suppressing, attenuating, or inhibiting one or more symptoms associated with T2D, comprising administering to a patient diagnosed with T2D a therapeutically effective amount of an isolated antagonistic antigen binding protein that specifically binds to the human glucagon receptor. In various embodiments, the one or more symptoms is selected from the group consisting of: glucose area under the curve (AUC) as determined by mixed meal tolerance test (MMTT), excess HbA1c levels, increased thirst and frequent urination, increased hunger, weight loss, fatigue, blurred vision, slow-healing sores or frequent infections, and areas of darkened skin.

In various embodiments, the isolated antibody or antigen-binding antibody fragment specifically binds to a human glucagon receptor with a dissociation constant (K_{D}) of at least about 1×10⁻⁷ M, at least about 1×10⁻⁸ M, at least about 1×10⁻⁹ M, at least about 1×10⁻¹⁰ M, at least about 1×10⁻¹¹ M, or at least about 1×10⁻¹² M.

In various embodiments, the isolated antagonistic antigen binding protein that specifically binds the human glucagon receptor will be admixed with a pharmaceutically acceptable excipient or carrier. In various embodiments, the pharmaceutical composition is formulated for administration via a route selected from the group consisting of subcutaneous injection, intraperitoneal injection, intramuscular injection, intrasternal injection, intravenous injection, intraarterial injection, intrathecal injection, intraventricular injection, intraurethral injection, intracranial injection, intrasynovial injection or via infusions.

### MODE(S) FOR CARRYING OUT THE INVENTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those commonly used and well known in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2nd ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those commonly used and well known in the art. Standard techniques are used for chemical syntheses, chemical analyses, pharmaceutical preparation, formulation, and delivery, and treatment of patients.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

### Definitions

The terms “glucagon inhibitor”, “glucagon suppressor” and “glucagon antagonist” are used interchangeably. Each is a molecule that detectably inhibits glucagon action or signaling. The inhibition caused by an inhibitor need not be complete so long as the inhibition is detectable using an assay that is recognized and understood in the art as being determinative of glucagon signaling inhibition.

An “antigen binding and antagonizing protein” is a protein comprising a portion that binds to an antigen and, optionally, a scaffold or framework portion that allows the antigen binding portion to adopt a conformation that promotes binding of the isolated antagonistic antigen binding protein to the antigen. Examples of antigen binding and antagonizing proteins include antibodies, antibody fragments (e.g., an antigen binding portion of an antibody), antibody derivatives, and antibody analogs. The isolated antagonistic antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the isolated antagonistic antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129 (2003); Roque et al., Biotechnol. Prog. 20:639-654 (2004). In addition, peptide antibody mimetics (“PAMs”) can be used, as well as scaffolds based on antibody mimetics utilizing fibronection components as a scaffold.

The term “human antibody” includes all antibodies that have one or more variable and/or constant regions derived from human immunoglobulin sequences. In one embodiment, all of the variable and constant domains are derived from human immunoglobulin sequences (a fully human antibody). These antibodies may be prepared in a variety of ways, examples of which are described below, including through the immunization with an antigen of interest of a mouse that is genetically modified to express antibodies derived from human heavy and/or light chain-encoding genes.

A “humanized antibody” has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human patient. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human patient, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

An isolated antagonistic antigen binding protein of the present disclosure, including an antibody, “specifically binds” to an antigen, such as the human glucagon receptor if it binds to the antigen with a high binding affinity as determined by a dissociation constant (Kd, or corresponding Kb, as defined below) value of 10⁻⁷ M or less. An isolated antagonistic antigen binding protein that specifically binds to the human glucagon receptor may be able to bind to glucagon receptors from other species as well with the same or different affinities.

An “epitope” is the portion of a molecule that is bound by an isolated antagonistic antigen binding protein (e.g., by an antibody). An epitope can comprise non-contiguous portions of the molecule (e.g., in a polypeptide, amino acid residues that are not contiguous in the polypeptide's primary sequence but that, in the context of the polypeptide's tertiary and quaternary structure, are near enough to each other to be bound by an antigen binding and antagonizing protein).

A “pharmaceutical composition” refers to a composition suitable for pharmaceutical use in an animal or human. A pharmaceutical composition comprises a pharmacologically and/or therapeutically effective amount of an active agent and a pharmaceutically acceptable carrier. “Pharmaceutically acceptable carrier” refers to compositions that do not produce adverse, allergic, or other untoward reactions when administered to an animal or a human. As used herein “pharmaceutically acceptable carrier” refers to any of the standard pharmaceutical carriers, vehicles, buffers, and carriers, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

The phrase “administering” or “cause to be administered” refers to the actions taken by a medical professional (e.g., a physician), or a person controlling medical care of a patient, that control and/or permit the administration of the agent(s)/compound(s) at issue to the patient. Causing to be administered can involve diagnosis and/or determination of an appropriate therapeutic regimen, and/or prescribing particular agent(s)/compounds for a patient. Such prescribing can include, for example, drafting a prescription form, annotating a medical record, and the like. Where administration is described herein, “causing to be administered” is also contemplated.

As used herein, the terms “co-administration”, “co-administered” and “in combination with”, referring to the isolated antagonistic antigen binding proteins of the invention and one or more other therapeutic agents, is intended to mean, and does refer to and include the following: simultaneous administration of such combination of isolated antagonistic antigen binding proteins of the invention and therapeutic agent(s) to an individual in need of treatment, when such components are formulated together into a single dosage form which releases said components at substantially the same time to said individual; substantially simultaneous administration of such combination of isolated antagonistic antigen binding proteins of the invention and therapeutic agent(s) to an individual in need of treatment, when such components are formulated apart from each other into separate dosage forms which are taken at substantially the same time by said individual, whereupon said components are released at substantially the same time to said individual; sequential administration of such combination of isolated antagonistic antigen binding proteins of the invention and therapeutic agent(s) to an individual in need of treatment, when such components are formulated apart from each other into separate dosage forms which are taken at consecutive times by said individual with a significant time interval between each administration, whereupon said components are released at substantially different times to said individual; and sequential administration of such combination of isolated antagonistic antigen binding proteins of the invention and therapeutic agent(s) to an individual in need of treatment, when such components are formulated together into a single dosage form which releases said components in a controlled manner whereupon they are concurrently, consecutively, and/or overlappingly released at the same and/or different times to said individual, where each part may be administered by either the same or a different route.

The terms “patient,” “individual,” and “patient” may be used interchangeably and refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In various embodiments, the patient can be a human (e.g., adult male, adult female, adolescent male, adolescent female, male child, female child) under the care of a physician or other health worker in a hospital, psychiatric care facility, as an outpatient, or other clinical context.

The terms “treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

As used herein, a “therapeutically effective amount” of an isolated antagonistic antigen binding protein that specifically binds the human glucagon receptor refers to an amount of such protein that, when provided to a patient in accordance with the disclosed and claimed methods effects one of the following biological activities: treats Type 2 diabetes; or reduces, suppresses, attenuates, or inhibits one or more symptoms of T2D.

As used herein and in the appended claims, the singular forms “a,” “or,” and “the” include plural referents unless the context clearly dictates otherwise. It is understood that aspects and variations of the disclosure described herein include “consisting” and/or “consisting essentially of” aspects and variation.

Reference to “about” a value or parameter herein includes (and describes) variations that are directed to that value or parameter per se. For example, description referring to “about X” includes description of “X”.

### Glucagon Receptor Antigen Binding and Antagonizing Proteins

Glucagon is a 29 amino acid hormone processed from its pre-pro-form in the pancreatic alpha cells by cell specific expression of prohormone convertase 2 (PC2), a neuroendocrine-specific protease involved in the intracellular maturation of prohormones and proneuropeptides (Furuta et al., J. Biol. Chem. 276: 27197-27202 (2001)). In vivo, glucagon is a major counter-regulatory hormone for insulin actions. During fasting, glucagon secretion increases in response to falling glucose levels. Increased glucagon secretion stimulates glucose production by promoting hepatic glycogenolysis and gluconeogenesis (Dunning and Gerich, Endocrine Reviews, 28:253-283 (2007)). Thus glucagon counterbalances the effects of insulin in maintaining normal levels of glucose in animals.

The biological effects of glucagon are mediated through the binding and subsequent activation of a specific cell surface receptor, the glucagon receptor. The glucagon receptor (GCGR) is a member of the secretin subfamily (family B) of G-protein-coupled receptors. The human GCGR is a 477 amino acid sequence GPCR and the amino acid sequence of GCGR is highly conserved across species (Mayo et al, Pharmacological Rev., 55:167-194, (2003)). The glucagon receptor is predominantly expressed in the liver, where it regulates hepatic glucose output, on the kidney, and on islet β-cells, reflecting its role in gluconeogenesis. The activation of the glucagon receptors in the liver stimulates the activity of adenyl cyclase and phosphoinositol turnover which subsequently results in increased expression of gluconeogenic enzymes including phosphoenolpyruvate carboxykinase (PEPCK), fructose-1,6-bisphosphatase (FBPase-1), and glucose-6-phosphatase (G-6-Pase). In addition, glucagon signaling activates glycogen phosphorylase and inhibits glycogen synthase. Studies have shown that higher basal glucagon levels and lack of suppression of postprandial glucagon secretion contribute to diabetic conditions in humans (Muller et al., N Eng J Med 283: 109-115 (1970)). As such, methods of controlling and lowering blood glucose by targeting glucagon production or function using a GCGR antagonist have been explored.

In various embodiments, the antigen binding and antagonizing proteins of the present disclosure may be selected to bind to membrane-bound glucagon receptors as expressed on cells, and inhibit or block glucagon signaling through the glucagon receptor. In various embodiments, the antigen binding and antagonizing proteins of the present disclosure specifically bind to the human glucagon receptor. In various embodiments, the antigen binding and antagonizing proteins binding to the human glucagon receptor may also bind to the glucagon receptors of other species. The polynucleotide and polypeptide sequences for several species of glucagon receptor (e.g., the mouse glucagon receptor (accession number AAH57988) or rat glucagon receptor (NM 172092)) are known (see, e.g., U.S. Pat. No. 7,947,809, herein incorporated by reference in its entirety for its specific teaching of polynucleotide and polypeptide sequences of a human, rat, mouse and cynomolgus glucagon receptor).

Methods of generating antibodies that bind to antigens such as the human glucagon receptor are known to those skilled in the art. For example, a method for generating a monoclonal antibody that binds specifically to a targeted antigen polypeptide may comprise administering to a mouse an amount of an immunogenic composition comprising the targeted antigen polypeptide effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the targeted antigen polypeptide. Once obtained, a hybridoma can be propagated in a cell culture, optionally in culture conditions where the hybridoma-derived cells produce the monoclonal antibody that binds specifically to targeted antigen polypeptide. The monoclonal antibody may be purified from the cell culture. A variety of different techniques are then available for testing an antigen/antibody interaction to identify particularly desirable antibodies.

Methods for making fully human antibodies have been described in the art. By way of example, a method for producing an antagonistic antigen binding protein that specifically binds to the human glucagon receptor comprises the steps of synthesizing a library of human antibodies on phage, screening the library with GCGR or an antibody binding portion thereof, isolating phage that bind GCGR, and obtaining the antibody from the phage. By way of another example, one method for preparing the library of antibodies for use in phage display techniques comprises the steps of immunizing a non-human animal comprising human immunoglobulin loci with GCGR or an antigenic portion thereof to create an immune response, extracting antibody-producing cells from the immunized animal; isolating RNA encoding heavy and light chains of antibodies of the disclosure from the extracted cells, reverse transcribing the RNA to produce cDNA, amplifying the cDNA using primers, and inserting the cDNA into a phage display vector such that antibodies are expressed on the phage.

Again, by way of example, recombinant antagonistic antigen binding protein that specifically binds to the human glucagon receptor can also be isolated by screening a recombinant combinatorial antibody library. Preferably the library is a scFv phage display library, generated using human V_{L} and V_{H} cDNAs prepared from mRNA isolated from B cells. Methods for preparing and screening such libraries are known in the art. Kits for generating phage display libraries are commercially available (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, WO 92/09690; Fuchs et al., Bio/Technology, 9:1370-1372 (1991); Hay et al., Hum. Antibod. Hybridomas, 3:81-85, 1992; Huse et al., Science, 246:1275-1281, 1989; McCafferty et al., Nature, 348:552-554, 1990; Griffiths et al., EMBO J., 12:725-734, 1993; Hawkins et al., J. Mol. Biol., 226:889-896, 1992; Clackson et al., Nature, 352:624-628, 1991; Gram et al., Proc. Natl. Acad. Sci. (U.S.A.), 89:3576-3580, 1992; Garrad et al., Bio/Technology, 9:1373-1377, 1991; Hoogenboom et al., Nuc. Acid Res., 19:4133-4137, 1991; and Barbas et al., Proc. Natl. Acad. Sci. (U.S.A.), 88:7978-7982, 1991), all incorporated herein by reference.

Human antibodies are also produced by immunizing a non-human, transgenic animal comprising within its genome some or all of human immunoglobulin heavy chain and light chain loci with a human IgE antigen, e.g., a XenoMouse™ animal (Abgenix, Inc./Amgen, Inc.—Fremont, Calif.). XenoMouse™ mice are engineered mouse strains that comprise large fragments of human immunoglobulin heavy chain and light chain loci and are deficient in mouse antibody production. See, e.g., Green et al., Nature Genetics, 7:13-21, 1994 and U.S. Pat. Nos. 5,916,771, 5,939,598, 5,985,615, 5,998,209, 6,075,181, 6,091,001, 6,114,598, 6,130,364, 6,162,963 and 6,150,584. XenoMouse™ mice produce an adult-like human repertoire of fully human antibodies and generate antigen-specific human antibodies. In some embodiments, the XenoMouse™ mice contain approximately 80% of the human antibody V gene repertoire through introduction of megabase sized, germline configuration fragments of the human heavy chain loci and kappa light chain loci in yeast artificial chromosome (YAC). In other embodiments, XenoMouse™ mice further contain approximately all of the human lambda light chain locus. See Mendez et al., Nature Genetics, 15:146-156, 1997; Green and Jakobovits, J. Exp. Med., 188:483-495, 1998; and WO 98/24893.

In various embodiments, the isolated antagonistic antigen binding protein of the present disclosure utilize an antibody or antigen binding antibody fragment thereof is a polyclonal antibody, a monoclonal antibody or antigen-binding fragment thereof, a recombinant antibody, a diabody, a chimerized or chimeric antibody or antigen-binding fragment thereof, a humanized antibody or antigen-binding fragment thereof, a fully human antibody or antigen-binding fragment thereof, a CDR-grafted antibody or antigen-binding fragment thereof, a single chain antibody, an Fv, an Fd, an Fab, an Fab′, or an F(ab′)_{2,} and synthetic or semi-synthetic antibodies.

In various embodiments, the isolated antagonistic antigen binding protein of the present disclosure utilize an antibody or antigen-binding fragment that binds to GCGR with a dissociation constant (K_{D}) of, e.g., at least about 1×10⁻⁷ M, at least about 1×10⁻⁸ M, at least about 1×10⁻⁹ M, at least about 1×10⁻¹⁰M, at least about 1×10⁻¹¹ M, or at least about 1×10⁻¹² M. In various embodiments, the isolated antagonistic antigen binding protein of the present disclosure utilize an antibody or antigen-binding fragment that binds to GCGR with a dissociation constant (K_{D}) in the range of, e.g., at least about 1×10⁻⁷ M to at least about 1×10⁻⁸ M, at least about 1×10⁻⁸ M to at least about 1×10⁻⁹ M, at least about 1×10⁻⁹ M to at least about 1×10⁻¹⁰ M, at least about 1×10⁻¹⁰ M to at least about 1×10⁻¹¹ M, or at least about 1×10⁻¹¹ M to at least about 1×10⁻¹² M.

Antibodies to the glucagon receptor have been described in, e.g., U.S. Pat. Nos. 5,770,445 and 7,947,809; European patent application EP2074149A2; EP patent EP0658200B1; U.S. patent publications 2009/0041784; 2009/0252727; 2013/0344538 and 2014/0335091; PCT publication WO2008/036341, and PCT publication WO2015/18968. In various embodiments of the present invention, the isolated antagonistic antigen binding protein is an anti-GCGR (“antagonistic”) antibody or antigen-binding fragment which comprises the polynucleotide and polypeptide sequences set forth in, e.g., U.S. Pat. No. 7,947,809, and 8,158,759, and PCT publication WO2015/189698, each herein incorporated by reference in its entirety for its specific teaching of polynucleotide and polypeptide sequences of various anti-GCGR antibodies or antigen-binding fragments.

In various embodiments, the isolated antagonistic antigen binding protein that specifically binds to the human glucagon receptor is a fully human IgG2 monoclonal antibody (the “REMD-477” antibody) which comprises a heavy chain variable region having the amino acid sequence of SEQ ID NO: 1:

(SEQ ID NO: 1)
QVQLVESGGGVVQPGRSLRLSCAASGFTFSSYGMHWVRQAPGKGLEWV
AVMWYDGSNKDYVDSVKGRFTISRDNSKNTLYLQMNRLRAEDTAVYYC
AREKDHYDILTGYNYYYGLDVWGQGTTVTVSS

and a light chain variable region having the amino acid sequence of SEQ ID NO: 2:

(SEQ ID NO: 2)
DIQMTQSPSSLSASVGDRVTITCRASQGIRNDLGWYQQKPGKAPKRLI
YAASSLQSGVPSRFSGSGSGTEFTLTISSVQPEDFVTYYCLQHNSNPL
TFGGGTKVEIK

In various embodiments, the antibody contains an amino acid sequence that shares an observed homology of, e.g., at least 70%, at least 75%, at least 80%, at least 85%, at least 90%, at least 91%, at least 92%, at least 93%, at least 94%, at least 95%, at least 96%, at least 97%, at least 98%, or at least 99% with the sequences of SEQ ID NOS: 1 or 2.

In various embodiments, the isolated antagonistic antigen binding protein that specifically binds to the human glucagon receptor may be an antibody which binds to the same epitope as the antibody comprising the heavy chain variable region sequence as set forth in SEQ ID NO: 1. In various embodiments, the isolated antagonistic antigen binding protein that specifically binds to the human glucagon receptor may be an antibody which competes with the antibody comprising the heavy chain variable region sequence as set forth in SEQ ID NO: 1. In various embodiments, the isolated antagonistic antigen binding protein that specifically binds to the human glucagon receptor may be an antibody which comprises at least one (such as two or three) CDRs of the heavy chain variable region sequence as set forth in SEQ ID NO: 1. In various embodiments, the isolated antagonistic antigen binding protein that specifically binds to the human glucagon receptor may be an antibody which binds to the same epitope as the antibody comprising the light chain variable region sequence as set forth in SEQ ID NO: 2. In various embodiments, the isolated antagonistic antigen binding protein that specifically binds to the human glucagon receptor may be an antibody which competes with the antibody comprising the light chain variable region sequence as set forth in SEQ ID NO: 2. In various embodiments, the isolated antagonistic antigen binding protein that specifically binds to the human glucagon receptor may be an antibody which comprises at least one (such as two or three) CDRs of the light chain variable region sequence as set forth in SEQ ID NO: 2.

In various embodiments, the isolated antagonistic antigen binding protein comprises an antibody comprising a heavy chain comprising CDR1 having the amino acid sequence of SEQ ID NO: 3, CDR2 having the amino acid sequence of SEQ ID NO: 4, and CDR3 having the amino acid sequence of SEQ ID NO: 5; and a light chain comprising CDR1 having the amino acid sequence of SEQ ID NO: 6, CDR2 having the amino acid sequence of SEQ ID NO: 7, and CDR3 having the amino acid sequence of SEQ ID NO: 8.

In vivo studies (both acute and chronic) using antagonizing GCGR antibodies have been conducted in normal mice, insulin-resistant diet-induced obesity (DIO) mice, and leptin-deficient ob/ob mice. Administration of antagonizing GCGR antibodies lowered blood glucose in a dose-dependent fashion without inducing hypoglycemia in normal, DIO, and ob/ob mice. In a study conducted in normal cynomolgus monkeys, a single SC injection of the REMD-477 analog, 2-59.1, resulted in improved glucose tolerance as well as increased glucagon and GLP-1 levels. In chronic rodent studies, the chimeric murine GCGR neutralizing antibody GR15c was used as a surrogate to assess the effects of blocking the glucagon pathway on hyperglucagonemia, alpha cell hyperplasia, and beta cell function. GR15c treatment caused dose-dependent hyperglucagonemia and minimal to mild alpha cell hyperplasia due to adaptive compensation for the reduced GCGR signal. There was no evidence in these studies of pancreatic alpha cell neoplastic transformation in mice treated with GR15c for as long as 18 weeks. Both treatment-induced hyperglucagonemia and alpha cell hyperplasia were reversible after treatment withdrawal for periods of 6 and 10 weeks. Importantly, pancreatic beta cell function was preserved, as demonstrated by improved glucose tolerance throughout the 18-week treatment period. In cell-based functional assays using recombinantly expressed GCGR from various species, including human, mouse, rat, and cynomolgus monkey, REMD-477 neutralizes glucagon-induced cellular response (as measured by changes in cyclic AMP [cAMP] levels).

Previous preclinical toxicology studies (by Amgen Inc) in rodents and monkeys have defined a favorable safety profile for REMD-477. No significant adverse effects were observed in toxicity studies of up to 9 months in duration in rats, and 3 months in monkeys at doses up to 300 mg/kg. The treatment-related changes such as increases in circulating glucagon and GLP-1 in these studies were considered secondary to glucagon receptor blockade and were thus expected and non-adverse.

The safety, tolerability, PK and PD of REMD-477 have been studied by Amgen Inc in a first-in-human (FIH) Phase 1a clinical study with administration of single subcutaneous (SC) or intravenous (IV) doses of REMD-477 in healthy adult volunteers. A total of 38 healthy volunteers completed the study, including 22 who received a single dose of REMD-477 SC administration (6 with 0.3 mg/kg, 5 with 1.0 mg/kg, and 11 with 2.5 mg/kg, respectively), 6 who received a single dose (0.3 mg/kg) REMD-477 IV administration, and 10 who received a placebo administration, respectively. The Phase 1 a study demonstrated a favorable safety profile of REMD-477, and all single SC doses of REMD-477 (0.3, 1.0 and 2.5 mg/kg) were well tolerated. All adverse events reported as treatment related for more than 1 individual were mild or moderate in severity. No deaths, no hypoglycemia, and no notable changes in ECG or vital signs were observed in the study. Small increases in aminotransferases (ALT and AST) without significant changes in total and free bilirubin or alkaline phosphatase were observed in 8 SC REMD-477-treated individuals (2 with 1 mg/kg and 6 with 2.5 mg/kg), with levels 2.4 x ULN (upper limit of normal). Four individuals who received REMD-477 0.3 mg/kg IV experienced increases in ALT and/or AST to levels ≤3.8×ULN. The ALT/AST increases occurred transiently within the period of approximately 1 to 3 weeks after administration of REMD-477, and did not reach the level of the “Drug induced liver injury (DILI)” per FDA's guidelines. These changes of ALT and AST were transient and reversible during the treatment, and/or during the post-treatment recovery. Following single dose SC administration, REMD-477 was slowly absorbed, with a median tₘₐₓ of 4 to 5 days post-dose. Exposure, as assessed by Cₘₐₓ and AUC₀₋ₜ, increased more than dose proportionally between the 0.3 mg/kg and 2.5 mg/kg SC doses. Mean residence time (MRT₀₋ₜ) increased with increasing dose and reached 317±63 hr at 2.5 mg/kg SC dose. The PK profile suggests that the effective half-life of REMD-477 approaches 2 weeks.

### Pharmaceutical Compositions

The isolated antagonistic GCGR binding proteins provided herein can be formulated by a variety of methods apparent to those of skill in the art of pharmaceutical formulation. Such methods may be found, for example, in Remington's Pharmaceutical Sciences, 19th Edition (Mack Publishing Company, 1995). The pharmaceutical compositions are generally formulated as sterile, substantially isotonic and in full compliance with all GMP regulations of the U.S. Food and Drug Administration.

Generally, isolated antagonistic GCGR binding proteins of the invention are suitable to be administered as a formulation in association with one or more pharmaceutically acceptable excipient(s), or carriers. Such pharmaceutically acceptable excipients and carriers are well known and understood by those of ordinary skill and have been extensively described (see, e.g., Remington's Pharmaceutical Sciences, 18th Edition, A. R. Gennaro, ed., Mack Publishing Company, 1990). The pharmaceutically acceptable carriers may be included for purposes of modifying, maintaining or preserving, for example, the pH, osmolarity, viscosity, clarity, color, isotonicity, odor, sterility, stability, rate of dissolution or release, adsorption or penetration of the composition. Such pharmaceutical compositions may influence the physical state, stability, rate of in vivo release, and rate of in vivo clearance of the polypeptide. Suitable pharmaceutically acceptable carriers include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCI, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute thereof. In one embodiment of the present disclosure, compositions may be prepared for storage by mixing the selected composition having the desired degree of purity with optional formulation agents (Remington's Pharmaceutical Sciences, supra) in the form of a lyophilized cake or an aqueous solution. Further, the therapeutic composition may be formulated as a lyophilizate using appropriate excipients such as sucrose. The optimal pharmaceutical composition will be determined by one of ordinary skill in the art depending upon, for example, the intended route of administration, delivery format, and desired dosage.

The pharmaceutical compositions of the invention are typically suitable for parenteral administration. As used herein, “parenteral administration” of a pharmaceutical composition includes any route of administration characterized by physical breaching of a tissue of a patient and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In various embodiments, the pharmaceutical composition is formulated for parenteral administration via a route selected from, e.g., subcutaneous injection, intraperitoneal injection, intramuscular injection, intrasternal injection, intravenous injection, intraarterial injection, intrathecal injection, intraventricular injection, intraurethral injection, intracranial injection, intrasynovial injection or via infusions.

When parenteral administration is contemplated, the therapeutic pharmaceutical compositions may be in the form of a pyrogen-free, parenterally acceptable aqueous solution comprising the desired isolated antagonistic GCGR binding protein in a pharmaceutically acceptable vehicle. A particularly suitable vehicle for parenteral injection is sterile distilled water in which a polypeptide is formulated as a sterile, isotonic solution, properly preserved. In various embodiments, pharmaceutical formulations suitable for injectable administration may be formulated in aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Optionally, the suspension may also contain suitable stabilizers or agents to increase the solubility of the compounds and allow for the preparation of highly concentrated solutions. Injectable formulations may be prepared, packaged, or sold in unit dosage form, such as in ampoules or in multi-dose containers containing a preservative. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

Any method for formulating and administering peptides, proteins, antibodies, and immunoconjugates accepted in the art may suitably be employed for administering the isolated antagonistic GCGR binding proteins of the present invention. In various embodiments, the REMD-477 of the present invention is produced by a certified GMP manufacturer, CMC Biologics, Seattle, USA, and supplied as a sterile, clear, colorless to slightly yellow frozen liquid Drug Product (DP) for subcutaneous administration. Each sterile vial is filled with 1 mL deliverable volume of 70 mg/mL REMD-477 formulated with 10 mM sodium acetate, 5% (w/v) sorbitol, 0.004% (w/v) polysorbate 20, pH 5.2.

### Therapeutic Methods of Use

In one aspect, the present invention relates to methods of treating Type 2 diabetes, comprising administering to a patient a therapeutically effective amount of an isolated antagonistic antigen binding protein that specifically binds to the human glucagon receptor. In various embodiments, the isolated antagonistic antigen binding protein comprises an antibody selected from a fully human antibody, a humanized antibody, a chimeric antibody, a monoclonal antibody, a polyclonal antibody, a recombinant antibody, an antigen-binding antibody fragment, a Fab, a Fab′, a Fab₂, a Fab′₂, a IgG, a IgM, a IgA, a IgE, a scFv, a dsFv, a dAb, a nanobody, a unibody, or a diabody. In various embodiments, the antibody is a fully human monoclonal antibody. In various embodiments, the fully human monoclonal antibody is REMD-477. In various embodiments, the method further comprises administering to the patient an antidiabetic medication selected from the group consisting of: biguanides (e.g., metformin), sulfonylureas, dipeptidyl- peptidase 4 inhibitors, sodium-glucose co-transporter 2 (SGLT2) inhibitors, α-glucosidase inhibitors, thiazolidinediones, bile acid sequestrants, amylin mimetics and incretin mimetics. The therapeutically effective doses of these antidiabetic medications have been well described in the art.

In another aspect, the present disclosure comprises a method for reducing, suppressing, attenuating, or inhibiting one or more symptoms associated with T2D, comprising administering to a patient diagnosed with T2D a therapeutically effective amount of an isolated antagonistic antigen binding protein that specifically binds to the human glucagon receptor. In various embodiments, the isolated antagonistic antigen binding protein comprises an antibody selected from a fully human antibody, a humanized antibody, a chimeric antibody, a monoclonal antibody, a polyclonal antibody, a recombinant antibody, an antigen-binding antibody fragment, a Fab, a Fab′, a Fab₂, a Fab′₂, a IgG, a IgM, a IgA, a IgE, a scFv, a dsFv, a dAb, a nanobody, a unibody, or a diabody. In various embodiments, the antibody is a fully human monoclonal antibody. In various embodiments, the fully human monoclonal antibody is REMD-477. In various embodiments, the method further comprises administering to the patient an antidiabetic medication selected from the group consisting of: biguanides (e.g., metformin), sulfonylureas, dipeptidyl- peptidase 4 inhibitors, sodium-glucose co-transporter 2 (SGLT2) inhibitors, α-glucosidase inhibitors, thiazolidinediones, bile acid sequestrants, amylin mimetics and incretin mimetics.

In another aspect, the present disclosure provides methods for treating a patient who is at risk of developing T2D (e.g., patients who have a greater than average risk of developing T2D) or patients with new onset adult T2D and insulin resistance or relative lack of insulin. These treatment methods can be carried out by (a) identifying a patient who is at risk (e.g., a heightened risk) of developing T2D and (b) administering to the patient a therapeutically effective amount of an isolated antagonistic antigen binding protein that specifically binds to the human glucagon receptor. In various embodiments, the isolated antagonistic antigen binding protein comprises an antibody selected from a fully human antibody, a humanized antibody, a chimeric antibody, a monoclonal antibody, a polyclonal antibody, a recombinant antibody, an antigen-binding antibody fragment, a Fab, a Fab′, a Fab₂, a Fab′₂, a IgG, a IgM, a IgA, a IgE, a scFv, a dsFv, a dAb, a nanobody, a unibody, or a diabody. In various embodiments, the antibody is a fully human monoclonal antibody. In various embodiments, the method further comprises administering to the patient an antidiabetic medication selected from the group consisting of: biguanides (e.g., metformin), sulfonylureas, dipeptidyl- peptidase 4 inhibitors, sodium-glucose co-transporter 2 (SGLT2) inhibitors, α-glucosidase inhibitors, thiazolidinediones, bile acid sequestrants, amylin mimetics and incretin mimetics.

In various embodiments, the T2D is refractory to treatment with an antidiabetic medication selected from the group consisting of: biguanides (e.g., metformin), sulfonylureas, dipeptidyl- peptidase 4 inhibitors, sodium-glucose co-transporter 2 (SGLT2) inhibitors, α-glucosidase inhibitors, thiazolidinediones, bile acid sequestrants, amylin mimetics and incretin mimetics.

### Dosing

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. The term “dosage unit form,” as used herein, refers to physically discrete units suited as unitary dosages for the patients to be treated; each unit contains a predetermined quantity of active compound calculated to produce the desired therapeutic effect in association with the required pharmaceutical carrier.

The precise dose of the isolated antagonistic antigen binding protein that specifically binds to the human glucagon receptor to be employed in the methods of the present disclosure will depend on the route of administration, and the seriousness of the disease or disorder, and should be decided according to the judgment of the practitioner and each patient's circumstances. It is to be noted that dosage values may include single or multiple doses, and that for any particular patient, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this disclosure may be based on a variety of factors, including the type of disease, the age, weight, sex, medical condition of the patient, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens are well-known in the relevant art and would be understood to be encompassed by the skilled artisan once provided the teachings disclosed herein.

An isolated antagonistic antigen binding protein that specifically binds the human glucagon receptor, in particular, the fully human antibodies of the disclosure, may be administered, e.g., once or more than once, at regular intervals over a period of time. In various embodiments, a fully human antibody is administered over a period of at least once a month or more, e.g., for one, two, or three months or even indefinitely. For treating chronic conditions, long-term treatment is generally most effective. However, for treating acute conditions, administration for shorter periods, e.g. from one to six weeks, may be sufficient. In general, the fully human antibody is administered until the patient manifests a medically relevant degree of improvement over baseline for the chosen indicator or indicators.

One example of therapeutic regimens provided herein comprise subcutaneous injection of an isolated antagonistic antigen binding protein once a week, or once every two weeks, at an appropriate dosage, to treat a condition in which blood glucose levels play a role. Weekly, bi-weekly or monthly administration of isolated antagonistic antigen binding protein would be continued until a desired result is achieved, e.g., the patient's symptoms subside. Treatment may resume as needed, or, alternatively, maintenance doses may be administered.

A patient's levels of blood glucose may be monitored before, during and/or after treatment with an isolated antagonistic antigen binding protein such as a human antibody, to detect changes, if any, in their levels. For some disorders, the incidence of elevated blood glucose may vary according to such factors as the stage of the disease. Known techniques may be employed for measuring glucose levels. Glucagon levels may also be measured in the patient's blood using know techniques, for example, ELISA.

A therapeutically effective dose can be estimated initially from cell culture assays by determining an IC₅₀. A dose can then be formulated in animal models to achieve a circulating plasma concentration range that includes the IC₅₀ as determined in cell culture. Such information can be used to more accurately determine useful doses in humans. Levels in plasma may be measured by, e.g., HPLC or immunoassays using the anti-idiotypic antibodies specific to the therapeutic drug. The exact composition, route of administration and dosage can be chosen by the individual physician in view of the patient's condition.

Dosage regimens can be adjusted to provide the optimum desired response (e.g., a therapeutic or prophylactic response). For example, a single bolus can be administered, several divided doses (multiple or repeat or maintenance) can be administered over time and the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian patients to be treated; each unit containing a predetermined quantity of active compound calculated to produce the desired therapeutic effect in association with the required pharmaceutical carrier. The specification for the dosage unit forms of the present disclosure will be dictated primarily by the unique characteristics of the antibody and the particular therapeutic or prophylactic effect to be achieved.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular patient, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this disclosure may be based on a variety of factors, including the type of disease, the age, weight, sex, medical condition of the patient, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens are well-known in the relevant art and would be understood to be encompassed by the skilled artisan once provided the teachings disclosed herein.

Toxicity and therapeutic index of the pharmaceutical compositions of the disclosure can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effective dose is the therapeutic index and it can be expressed as the ratio LD₅₀/ED₅₀. Compositions that exhibit large therapeutic indices are generally preferred.

In various embodiments, single or multiple administrations of the pharmaceutical compositions are administered depending on the dosage and frequency as required and tolerated by the patient. In any event, the composition should provide a sufficient quantity of at least one of the isolated antagonistic antigen binding protein disclosed herein to effectively treat the patient. The dosage can be administered once but may be applied periodically until either a therapeutic result is achieved or until side effects warrant discontinuation of therapy.

The dosing frequency of the administration of the isolated antagonistic antigen binding protein pharmaceutical composition depends on the nature of the therapy and the particular disease being treated. The patient can be treated at regular intervals, such as weekly, bi-weekly or monthly, until a desired therapeutic result is achieved. Exemplary dosing frequencies include, but are not limited to: once weekly without break; once weekly, every other week; once every 2 weeks; once every 3 weeks; weakly without break for 2 weeks, then monthly; weakly without break for 3 weeks, then monthly; monthly; once every other month; once every three months; once every four months; once every five months; once every six months; once every seven months; once every eight months; once every nine months; once every ten months; once every eleven months; or yearly.

In various embodiments, REMD-477 is administered to the patient at a dosage (e.g., at a weekly dosage) included in any of the following ranges: about 0.1 to about 0.2 mg/kg, about 0.2 to about 0.4 mg/kg, about 0.4 to about 0.6 mg/kg, about 0.6 to about 0.8 mg/kg, about 0.8 to about 1.0 mg/kg, about 1.0 to about 1.2 mg/kg, about 1.2 to about 1.4 mg/kg, about 1.4 to about 1.6 mg/kg, about 1.6 to about 1.8 mg/kg, about 1.8 to about 2.0 mg/kg, about 2.0 to about 2.2 mg/kg, about 2.2 to about 2.4 mg/kg, about 2.4 to about 2.6 mg/kg, about 2.6 to about 2.8 mg/kg, and about 2.8 to about 3.0 mg/kg. In various embodiments, REMD-477 is administered to the patient at a weekly dosage selected from the group consisting of .1 mg/kg, .2 mg/kg, .3 mg/kg, .4 mg/kg, .5 mg/kg, .6 mg/kg, .7 mg/kg, .8 mg/kg, .9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, and 3.0 mg/kg. In various embodiments, REMD-447 is administered at .2 mg/kg. In various embodiments, REMD-447 is administered at .4 mg/kg. In various embodiments, REMD-447 is administered at .6 mg/kg. In various embodiments, REMD-447 is administered at 1.0 mg/kg.

In various embodiments, REMD-477 is administered to the patient at a dosage (e.g., at a weekly dosage) included in any of the following ranges: about 5 to about 15 mg, about 15 to about 30 mg, about 30 to about 45 mg, about 45 to about 60 mg, about 60 to about 75 mg, about 75 to about 90 mg, about 90 to about 105 mg, about 105 to about 120 mg, about 120 to about 135 mg, about 135 to about 150 mg, about 150 to about 165 mg, about 165 to about 180 mg, about 180 to about 195 mg, and about 195 to about 210 mg. In various embodiments, REMD-477 is administered to the patient at a weekly dosage selected from the group consisting of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 200 mg, 205 mg, and 210 mg.

In various embodiments, the methods will further comprise administration of one or more antidiabetic medications to the patient. The list of exemplary antidiabetic medications includes, but is not limited to, biguanides (e.g., metformin), sulfonylureas, dipeptidyl- peptidase 4 inhibitors, sodium-glucose co-transporter 2 (SGLT2) inhibitors, α-glucosidase inhibitors, thiazolidinediones (e.g., rosiglitazone), bile acid sequestrants, amylin mimetics and incretin mimetics. In various embodiments, the antidiabetic medication is metformin and is administered in a dosage range of about 1000 to about 3000 mg/day. In various embodiments, injections of insulin may be added to the antidiabetic medication administration.

In various embodiments, REMD-447 is administered at about 0.1 to about 0.2 mg/kg, about 0.2 to about 0.4 mg/kg, about 0.4 to about 0.6 mg/kg, about 0.6 to about 0.8 mg/kg, about 0.8 to about 1.0 mg/kg, about 1.0 to about 1.2 mg/kg, about 1.2 to about 1.4 mg/kg, about 1.4 to about 1.6 mg/kg, about 1.6 to about 1.8 mg/kg, about 1.8 to about 2.0 mg/kg, about 2.0 to about 2.2 mg/kg, about 2.2 to about 2.4 mg/kg, about 2.4 to about 2.6 mg/kg, about 2.6 to about 2.8 mg/kg, and about 2.8 to about 3.0 mg/kg weekly and metformin is administered in a dosage range of about 1000 to about 3000 mg/day. In various embodiments, REMD-447 is administered at .2 mg/kg weekly and metformin is administered in a dosage range of about 1000 to about 3000 mg/day. In various embodiments, REMD-447 is administered at .4 mg/kg weekly and metformin is administered in a dosage range of about 1000 to about 3000 mg/day. In various embodiments, REMD-447 is administered at .6 mg/kg weekly and metformin is administered in a dosage range of about 1000 to about 3000 mg/day. In various embodiments, REMD-447 is administered at 1.0 mg/kg weekly and metformin is administered in a dosage range of about 1000 to about 3000 mg/day.

These various combination therapies may provide a “synergistic effect”, i.e., the effect achieved when the active ingredients used together is greater than the sum of the effects that results from using the compounds separately.

The following examples are provided to describe the invention in further detail.

### EXAMPLE 1

This example is a Phase 1 b/2a randomized, placebo-controlled, double-blind, dose escalation study to evaluate safety, tolerability, pharmacokinetics and pharmacodynamics of single and repeated subcutaneous doses of REMD-477 in patients with Type 2 Diabetes Mellitus. The primary objectives of the study are to characterize the safety and tolerability of REMD-477 in patients with T2D, following single and repeated subcutaneous (SC) administration. The secondary objectives of the study are to characterize the pharmacokinetic (PK) profile of REMD-477 following single and repeated SC administration in Type 2 diabetic patients; to characterize changes in fasting glucose and insulin levels following single and repeated SC doses of REMD-477, and in response to mixed meal tolerance test (MMTT) following single and repeated SC doses of REMD-477; and to characterize the effects of single and repeated SC doses of REMD-477 on immunogenicity of REMD-477, liver function tests (ALT, AST, ALP and total and direct bilirubin), and on pancreatic markers (amylase and lipase). Exploratory objectives of the study are to explore the effects of repeated SC doses of REMD-477 on chronic markers of glycemic control (i.e., fructosamine and HbA1c); to explore the effects of single and repeated doses of REMD-477 on circulating levels of glucagon, C-peptide, active and total glucagon-like peptide 1 (GLP-1); and to explore the effects of single and repeated doses of REMD-477 on the homeostatic model assessment of insulin resistance (HOMA-IR) and β-cell function (HOMA-β) and on the model- derived dynamic indices of IR and IS (insulin resistance and insulin secretion).

The study will be conducted at multiple sites in the United States and will enroll approximately 72 patients (no more than 84 patients will be enrolled) with Type 2 diabetes who are either treatment-naïve, controlled with diet and exercise or treated with oral antidiabetic medications. Oral antidiabetic medications, such as metformin, sulfonylureas, dipeptidyl-peptidase 4 inhibitors, sodium-glucose co-transporter 2 (SGLT2) inhibitors, or α-glucosidase inhibitors, will require at least a 1 week washout prior to randomization (Day 1). The study will consist of a dose escalation phase (Part A) followed by an adoptive dose cohort phase (Part B). Including the 28-day screening period, Lead-in Period of up to 4 weeks, study drug treatment period of 84 days and a safety follow-up period of 56 days, the maximum patient participation time is approximately 196 days.

Eligible patients are 18 years and older, male or female. The eligibility inclusion criteria includes: Females of non-child bearing potential must be ≥1 year post-menopausal (confirmed by a serum follicle-stimulating hormone (FSH) levels ≥40 mIU/mL) or documented as being surgically sterile, and females of child bearing potential must use two medically acceptable methods of contraception; male patients must be willing to use medically acceptable contraception or abstain from sexual intercourse during treatment and for 2 months after the last administration of REMD-477; normal or clinically-acceptable physical examination, laboratory test values, and 12-lead ECG (reporting heart rate and PR, QRS, QT, and QT_{c}F) at screening; body mass index between 23 and 40 kg/m², inclusive, at screening; diagnosed with Type 2 diabetes as defined by the current American Diabetes Association (ADA) criteria (e.g., HbA1c ≥6.5%, or fasting plasma glucose ≥126 mg/dL (7.0 mmol/L), or 2-hour plasma glucose ≥200 mg/dL (11.1 mmol/L) during an oral glucose tolerance test, or a random plasma glucose ≥200 mg/dL (11.1 mmol/L) with classic symptoms of hyperglycemia or hyperglycemic crisis); treatment-naive, controlled with diet and exercise, or treated with oral antidiabetic medications and willing to wash-out and discontinue oral medications during the study. Oral antidiabetic medications that are prescribed (such as metformin, sulfonylureas, dipeptidyl-peptidase 4 inhibitors, SGLT2 inhibitors, or a-glucosidase inhibitors,) and/or over-the-counter medications or nutritional/herbal supplements for diabetics (such as St. John's wort, etc.) will require at least a1-week washout prior to randomization (Day 1); fasting plasma glucose 126 - 270 mg/dL (7-15 mM), inclusive, at screening and at re-test on Day -1; screening HbA1c of 7.0-10% inclusive for patients not currently taking any oral antidiabetic medications, or 6.5-9.5% inclusive for patients receiving acceptable oral antidiabetic medications.

The eligibility exclusion criteria includes: history or evidence of clinically-significant disorder, condition, or disease that, in the opinion of the Investigator, would pose a risk to patient safety or interfere with the study evaluation, procedures, or completion; known sensitivity to mammalian-derived drug preparations, or to humanized or human antibodies; history of drug or alcohol abuse within the last 6 months or a positive illegal drug urine test result (inclusive of medical marijuana use for diabetes) at screening or Day-1. The use of medical marijuana requires at least a 3 weekwash-out; history or family history of pancreatic neuroendocrine tumors or multiple endocrine neoplasia; history or family history of pheochromocytoma; known or suspected susceptibility to infectious disease (eg, taking immunosuppressive agents or has a documented inherited or acquired immunodeficiency); positive for human immunodeficiency virus (HIV) antibodies, hepatitis B surface antigen (HbsAg), or hepatitis C antibodies (HepC Ab); participation in an investigational drug or device trial within 30 days of screening or within 5 times the half-life of the investigational agent in the other clinical study, if known, whichever period is longer; blood donor, or blood loss>300 mL, within 30 days of Day 1; unavailable for follow-up assessment, or evidence of concerns with patient's compliance with the protocol procedures; any other conditions that might reduce the chance of obtaining data required by the protocol or that might compromise the ability to give truly informed consent; recent use (6 weeks prior to Screening) of thiazolidinediones, >half-maximal dose sulfonylurea agent therapy, or any injectable antidiabetic agents (exenatide and other injectable GLP-1 agonists, insulin and insulin analogs, etc.); other gastrointestinal, cardiac, renal and CNS (i.e. hypoglycemia unawareness) conditions specific to diabetes that would pose additional risk to patient's safety or interfere with the study evaluation, procedures or completion; lipid panel profiles of non-HDL-C (total cholesterol minus HDL-C) >219 mg/dL, LDL-C >189 mg/dL, HDL outside the normal reference ranges of the central laboratory, and/or fasting triglycerides >499 mg/dL; and female patient is pregnant or breastfeeding.

After informed consent, all screening procedures and tests to establish eligibility will be performed on all patients within a period of 28 days before initiating a Lead-in Period. Screening procedures include: medical history review, physical exam, body weight, height, urinary pregnancy test (women), urine drug and alcohol test, 12-lead ECG, vital signs, metabolic panel, hematology, urinalysis, fasting glucose, liver and pancreatic enzyme tests, fructosamine and HbA1c. The Lead-in Period applies to all patients and will include a minimum duration of a week when the patient begins to measure daily finger stick blood glucose levels (using the study provided glucometer) for at least twice per day: 1) after an overnight fasting in the morning and 2) at least 3 hours post last meal before bedtime. This period also includes an optional 1 week to 4 weeks washout for patients who are taking oral antidiabetic medications at the time of Screening. At the end of the Lead-in Period prior to dosing, specific Screening tests and procedures will be repeated within 7 days of Day-1 (Day-5±2) to ensure that the patients continue to meet study eligibility. Randomization to REMD-477 or placebo will be based on a randomization schedule prepared by the CRO's designated unblinded biostatistician and provided to each of the unblinded site pharmacist or designee before the start of the study.

REMD-477 will be supplied as single-use glass vials and formulated with 10 mM sodium acetate, pH 5.2, 5% (w/v) sorbitol, 0.004% polysorbate 20 at a protein concentration of 70 mg/mL. The placebo will be supplied as single-use glass vials and formulated with 10 mM sodium acetate, pH 5.2, 5% (w/v) sorbitol, 0.004% polysorbate 20. All REMD-477 and placebo injections will be given in the same volume for the same cohort. REMD-477 and placebo will be administered by SC injection. A single vial and a single injection site will be used for each dose administration. Each SC dose will be administered to the patient's anterior abdominal wall and will be no more than 1 mL. Alternating (left or right) sites on the patient's anterior abdominal wall and at a distance of at least 2 cm apart will be used for subsequent SC dosing.

The Dose-Escalation Stage (Part A) will consist of Cohorts 1-4 (n=12/Cohort) sequentially treated with REMD-477 (T) at escalating doses of 14 mg (Cohort-1), 28 mg (Cohort-2), 42 mg (Cohort-3) and 70 mg (Cohort-4) or Placebo (P), in a 3:1 randomization (T:P) for each cohort. Each cohort will receive one single SC injection (Dose-1) on Day 1, and safety and PK/PD measurements will be obtained for the next 4 weeks (week-1 through week-4), followed by 8 weekly SC doses (Dose-2 starting on day 29 (±1) and the last dose (Dose-9) on day 78 (±1). Patients will be asked to check finger stick blood glucose twice daily (in the morning with 8 hours fasting, and at least 3 hours post last meal before bedtime), and at additional times if deemed necessary by the Principal Investigator. All patients are to keep a daily log to record daily blood glucose levels. Adverse events and concomitant medications will be collected throughout the study when reported. Any rescue medications and use of meals/food to manage hypoglycemic sensations or events will be reviewed carefully with the patient and recorded accordingly. If there is a clinically significant laboratory abnormality or adverse event in need of monitoring, patients will be followed until resolution of the abnormality or adverse event or until it is considered stable.

Dose Level Review Meetings (DLRM) to determine whether to dose escalate from Cohort N to Cohort N+1 will be held when at least 9 patients (75% of the patients) in the cohort have completed the Day 57 safety assessments (i.e, patients have received Dose-5 (completion of Week-8 Visit) and its 1-week post-dose safety monitoring). All available safety data (including demographics, adverse events, vital signs, ECGs and safety labs) will be reviewed via teleconference by DLRM committee. At least two thirds of the voting DLRM members must agree to the decision to dose escalation. Cohort N+1 will be open for enrollment, after all of the patients in Cohort N have been enrolled. For the adoptive dose cohort phase (Part B), the decision to initiate Cohort- 5 will be triggered after at least 9 patients (75% of the patients) in Cohort-4 or the highest tolerated dose cohort have completed the Day 57 safety assessments. The decision to initiate Cohort-5, the dose selection and dosing intervals for Cohort-5 will be determined by the DLRM Committee, after review of cumulative safety data and all available PK, and PD data from all preceding cohorts. Cohort 5 will be opened for enrollment, after all patients in Cohort 4 have been enrolled.

An adoptive dose cohort phase (Part B) (Cohort-5) will consist of 24 patients, who will be randomized to REMD-447 or placebo in a 3:1 ratio. The dose of REMD-477 (up to 70 mg SC) and dose interval will be determined by the DLRM committee based on a careful review of cumulative safety data and all available PK, and PD data obtained from Part A. The decision to dose escalate will be made by the DLRM committee on the basis of a blinded review of all available cumulative study data. For Part A, the decision to dose escalate from Cohort N to Cohort N+1 will proceed after at least 9 patients (75% of all patients) in Cohort N have received Dose-5 (completion of Week-8 Visit) and its 1-week post-dose safety monitoring (completion of the Day 57 safety assessments). Cohort N+1 will be open for enrollment, after all of the patients in Cohort N have been enrolled. For Part B, the decision to initiate Cohort-5 will be triggered after at least 9 patients (75% of the patients) in Cohort-4 or the highest tolerated dose cohort have completed the Day 57 safety assessments. The decision to initiate Cohort-5, the dose selection and dosing intervals for Cohort-5 will be determined by the DLRM committee, after review of cumulative safety data and all available PK, and PD data from all preceding cohorts. Cohort 5 will be opened for enrollment, after all patients in Cohort 4 have been enrolled. The DLRM committee may choose to postpone the decision to initiate Cohort-5 until more safety, PK and PD data become available from the proceeding cohort(s).

Study endpoints , Subsets, and Covariates are as follows: 1) Primary Endpoints: patient incidence of adverse events and clinically relevant changes in medical history, physical examination, laboratory safety values, and ECGs; 2) Secondary Endpoints: pharmacokinetic parameters including Cmax, AUC, clearance, and half-life (t½) after single and repeated SC doses; changes in fasting plasma glucose and insulin after single and repeated SC doses; changes in glucose and insulin AUC following MMTT after repeated SC doses; incidence of REMD-477 neutralizing and non-neutralizing antibodies; incidence of elevated alanine transaminase (ALT) or aspartate transaminase (AST) values >3× the upper limit of normal (ULN), with concomitant >2× increases in alkaline phosphatase (ALP) and/or >2× total bilirubin. Geometric mean ratio to baseline over time of AST, ALT, ALP and total bilirubin; incidence and severity of elevated amylase and lipase values at >2.5× ULN after study treatment; and 3) Exploratory Endpoints: changes in hemoglobin A1C (HbA1c) and fructosamine levels following repeated SC doses of REMD-477; changes in glucagon, C-peptide, active and total GLP-1 following single and repeated SC doses of REMD-477; and changes in the HOMA-IR and HOMA-β values and on the model-derived dynamic indices of IR and IS (insulin resistance and insulin secretion) following single (Day 29±1) and repeated SC doses (Day 85±1) of REMD-477, in comparison to the baseline (Day-1) values.

Patients receiving all 9 doses in any Cohort will be followed for 8 weeks after the last dose and will complete study evaluations. The EOS visit will occur on Day 141. Patients who are positive for anti-REMD-477 neutralizing antibodies at the end of study will have subsequent blood samples drawn 30 days after testing positive and every 3 months thereafter until the results for neutralizing antibodies are negative or become stabilized. All adverse events and use of concomitant medications will be documented for the duration of the study up to and including the EOS visit. If there is a clinically significant clinical or laboratory abnormality in need of monitoring, patients will be followed until resolution of the abnormality or until it is considered stable.

It is envisioned that treatment using REMD-477 at the optimal doses described herein will result in a reduction in fasting blood glucose, a reduction in glucose area under the curve (AUC) as determined by mixed meal tolerance test (MMTT), a robust and sustained lowering of hemoglobin A1C (HbA1c) levels, and increases in serum glucagon and serum GLP-1 levels following single and repeated SC doses of REMD-477. And importantly, the REMD-477 will be safe and well tolerated.

### EXAMPLE 2

This example describes a Double Blind, Placebo-Controlled Study evaluating the safety, tolerability, pharmacokinetics and pharmacodynamics of REMD-477 administered once weekly to patients with Type 2 Diabetes Mellitus being treated with metformin. Eligible patients are 18 years and older, male or female, having Type 2 Diabetes Mellitus, having HbA1 c levels greater than or equal to 7.5% and less than or equal to 10.5%, and on a stable dose of oral metformin (at least 1000 mg/day and up to 3000 mg/day) for a minimum of 3 months prior to screening evaluation and will be required to continue their stable dose of metformin throughout the study.

The study will consist of two Cohorts (n=12/Cohort) sequentially treated with REMD-477 (T) at a dose of 35 mgs (Cohort-1) or 42 mgs (Cohort-2) or Placebo (P), in a 3:1 randomization (T:P) for each cohort. Each cohort will receive 12 weekly SC doses of REMD-477 while continuing on their stable daily dose of metformin (at least 1000 mg/day and up to 3000 mg/day). Patients will be asked to check finger stick blood glucose twice daily (in the morning with 8 hours fasting, and at least 3 hours post last meal before bedtime), and at additional times if deemed necessary by the Principal Investigator. All patients are to keep a daily log to record daily blood glucose levels. Adverse events and concomitant medications will be collected throughout the study when reported. Any rescue medications and use of meals/food to manage hypoglycemic sensations or events will be reviewed carefully with the patient and recorded accordingly. If there is a clinically significant laboratory abnormality or adverse event in need of monitoring, patients will be followed until resolution of the abnormality or adverse event or until it is considered stable.

It is envisioned that this study will demonstrate that combination therapy using REMD-447 at the optimal doses described herein will achieve robust and sustained, statistically significant improvements in hemoglobin A1c (HbA1c) levels and other measures of glucose control at the end of treatment, as compared to placebo. And importantly, the REMD-477 will be safe and well tolerated. As such, combination therapy comprising REMD-477 and metformin represents a major advancement in the treatment of type 2 diabetes.

All of the articles and methods disclosed and claimed herein can be made and executed without undue experimentation in light of the present invention. While the articles and methods of this invention have been described in terms of various embodiments, it will be apparent to those of skill in the art that variations may be applied to the articles and methods without departing from the spirit and scope of the invention. All such variations and equivalents apparent to those skilled in the art, whether now existing or later developed, are deemed to be within the spirit and scope of the invention as defined by the appended claims. All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes. The invention illustratively described herein suitably may be practiced in the absence of any element(s) not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by various embodiments and optional features, modification and variation of the concepts herein disclosed may be resorted to by those skilled in the art, and that such modifications and variations are considered to be within the scope of this invention as defined by the appended claims.

### SEQUENCE LISTINGS

The amino acid sequences listed in the accompanying sequence listing are shown using standard three letter code for amino acids, as defined in 37 C.F.R. 1.822.

SEQ ID NO: 1 is the amino acid sequence encoding the heavy chain variable region of an antagonistic antibody that specifically binds to the human glucagon receptor.

SEQ ID NO: 2 is the amino acid sequence encoding the light chain variable region of an antagonistic antibody that specifically binds to the human glucagon receptor.

SEQ ID NO: 3 is the amino acid sequence of a heavy chain CDR1 of an antagonistic antibody that specifically binds to the human glucagon receptor.

SEQ ID NO: 4 is the amino acid sequence of a heavy chain CDR2 of an antagonistic antibody that specifically binds to the human glucagon receptor.

SEQ ID NO: 5 is the amino acid sequence of a heavy chain CDR3 of an antagonistic antibody that specifically binds to the human glucagon receptor.

SEQ ID NO: 6 is the amino acid sequence of a light chain CDR1 of an antagonistic antibody that specifically binds to the human glucagon receptor.

SEQ ID NO: 7 is the amino acid sequence of a light chain CDR2 of an antagonistic antibody that specifically binds to the human glucagon receptor.

SEQ ID NO: 8 is the amino acid sequence of a light chain CDR3 of an antagonistic antibody that specifically binds to the human glucagon receptor.

### SEQUENCE LISTINGS

SEQ ID NO: 1-13 amino acid sequence encoding the heavy chain variable region of an antagonistic antibody that specifically binds to the human glucagon receptor.

QVQLVESGGGVVQPGRSLRLSCAASGFTFSSYGMHWVRQAPGKGLEWV
AVMWYDGSNKDYVDSVKGRFTISRDNSKNTLYLQMNRLRAEDTAVYYC
AREKDHYDILTGYNYYYGLDVWGQGTTVTVSS

SEQ ID NO: 2—amino acid sequence encoding the light chain variable region of an antagonistic antibody that specifically binds to the human glucagon receptor.

DIQMTQSPSSLSASVGDRVTITCRASQGIRNDLGWYQQKPGKAPKRLI
YAASSLQSGVPSRFSGSGSGTEFTLTISSVQPEDFVTYYCLQHNSNPL
TFGGGTKVEIK

SEQ ID NO: 3—amino acid sequence of a heavy chain CDR1 of an antagonistic antibody that specifically binds to the human glucagon receptor.

SYGMH

SEQ ID NO: 4—amino acid sequence of a heavy chain CDR2 of an antagonistic antibody that specifically binds to the human glucagon receptor.

VMWYDGSNKDYVDSVKG

SEQ ID NO: 5—amino acid sequence of a heavy chain CDR3 of an antagonistic antibody that specifically binds to the human glucagon receptor.

EKDHYDILTGYNYYYGLDV

SEQ ID NO: 6—amino acid sequence of a light chain CDR1 of an antagonistic antibody that specifically binds to the human glucagon receptor.

RASQGIRNDLG

SEQ ID NO: 7—amino acid sequence of a light chain CDR2 of an antagonistic antibody that specifically binds to the human glucagon receptor.

AASSLQS

SEQ ID NO: 8—amino acid sequence of a light chain CDR3 of an antagonistic antibody that specifically binds to the human glucagon receptor.

LQHNSNPLT

1-19. (canceled)

20. A method for treating a patient diagnosed with Type 2 Diabetes Mellitus (T2D) comprising administering to the patient a therapeutically effective amount of an isolated antagonistic antigen binding protein that specifically binds to the human glucagon receptor; wherein the therapeutically effective amount of the isolated antagonistic antigen binding protein is from about 0.1 mg/kg to about 3.0 mg/kg.

21. A method according to claim 20, wherein the isolated antagonistic antigen binding protein comprises an isolated antagonistic antibody selected from the group consisting of a fully human antibody, a humanized antibody, a chimeric antibody, a monoclonal antibody, a polyclonal antibody, a recombinant antibody, an antigen-binding antibody fragment, a Fab, a Fab′, a Fab₂, a Fab′₂, a IgG, a IgM, a IgA, a IgE, a scFv, a dsFv, a dAb, a nanobody, a unibody, a diabody, and a hemibody.

22. A method according to claim 21, wherein the therapeutically effective amount of the isolated antagonistic antibody is selected from the group consisting of: about 0.1 to about 0.2 mg/kg, about 0.2 to about 0.4 mg/kg, about 0.4 to about 0.6 mg/kg, about 0.6 to about 0.8 mg/kg, about 0.8 to about 1.0 mg/kg, about 1.0 to about 1.2 mg/kg, about 1.2 to about 1.4 mg/kg, about 1.4 to about 1.6 mg/kg, about 1.6 to about 1.8 mg/kg, about 1.8 to about 2.0 mg/kg, about 2.0 to about 2.2 mg/kg, about 2.2 to about 2.4 mg/kg, about 2.4 to about 2.6 mg/kg, about 2.6 to about 2.8 mg/kg, and about 2.8 to about 3.0 mg/kg.

23. A method according to claim 22, wherein the therapeutically effective amount of the isolated antagonistic antibody is selected from the group consisting of: .2 mg/kg, .4 mg/kg, .6 mg/kg, and 1.0 mg/kg.

24. A method according to claim 23, wherein the therapeutically effective amount of the isolated antagonistic antibody is administered weekly.

25. A method according to claim 24, wherein the therapeutically effective amount of the isolated antagonistic antibody is administered subcutaneously.

26. A method according to claim 25, wherein the systemic administration is selected from:
intravenous injection, intramuscular injection, subcutaneous injection, intraperitoneal injection, transdermal injection, intraarterial injection, intrasternal injection, intrathecal injection, intraventricular injection, intraurethral injection, intracranial injection, intrasynovial injection or via infusions.

27. A method according to claim 20, wherein the therapeutically effective amount of the isolated antagonistic antibody is selected from the group consisting of: about 5 to about 15 mg, about 15 to about 30 mg, about 30 to about 45 mg, about 45 to about 60 mg, about 60 to about 75 mg, about 75 to about 90 mg, about 90 to about 105mg.

28. A method according to claim 27, wherein the therapeutically effective amount of the isolated antagonistic antigen binding protein is selected from the group consisting of: 14 mg, 28 mg, 42 mg, and 70 mg.

29. A method according to claim 20, wherein the isolated antagonistic antibody comprises a heavy chain comprising a CDR1 having the amino acid sequence of SEQ ID NO: 3, a CDR2 having the amino acid sequence of SEQ ID NO: 4, and a CDR3 having the amino acid sequence of SEQ ID NO: 5; and comprising a light chain comprising a CDR1 having the amino acid sequence of SEQ ID NO: 6, a CDR2 having the amino acid sequence of SEQ ID NO: 7, and a CDR3 having the amino acid sequence of SEQ ID NO: 8.

30. A method according to claim 29, wherein the isolated antagonistic antibody is a fully human antibody.

31. A method according to claim 30, wherein the fully human antibody comprises a heavy chain variable region having the amino acid sequence of SEQ ID NO: 1 and a light chain variable region having the amino acid sequence of SEQ ID NO:2.

32. A method according to claim 20, further comprising administering to the patient an antidiabetic medication selected from the group consisting of: biguanides (e.g., metformin), sulfonylureas, dipeptidyl-peptidase 4 inhibitors, sodium-glucose co-transporter 2 (SGLT2) inhibitors, α-glucosidase inhibitors, thiazolidinediones, bile acid sequestrants, amylin mimetics and incretin mimetics.

33. A method according to claim 32, wherein the isolated antagonistic antigen binding protein is administered weekly, and the antidiabetic medication is administered daily.

34. A method according to claim 33, wherein the isolated antagonistic antigen binding protein is a fully human antibody comprising a heavy chain variable region having the amino acid sequence of SEQ ID NO: 1 and a light chain variable region having the amino acid sequence of SEQ ID NO: 2;
and wherein the antidiabetic medication is metformin.

35. A method according to claim 34, wherein the therapeutically effective amount of the isolated antagonistic antibody is selected from the group consisting of: about 0.1 to about 0.2 mg/kg, about 0.2 to about 0.4 mg/kg, about 0.4 to about 0.6 mg/kg, about 0.6 to about 0.8 mg/kg, about 0.8 to about 1.0 mg/kg, about 1.0 to about 1.2 mg/kg, about 1.2 to about 1.4 mg/kg, about 1.4 to about 1.6 mg/kg, about 1.6 to about 1.8 mg/kg, about 1.8 to about 2.0 mg/kg, about 2.0 to about 2.2 mg/kg, about 2.2 to about 2.4 mg/kg, about 2.4 to about 2.6 mg/kg, about 2.6 to about 2.8 mg/kg, and about 2.8 to about 3.0 mg/kg.

36. A method of reducing, suppressing, attenuating, or inhibiting one or more symptoms associated with T2D, comprising administering to a patient diagnosed with T2D a therapeutically effective amount of an isolated antagonistic antigen binding protein that specifically binds to the human glucagon receptor; wherein the therapeutically effective amount of the isolated antagonistic antigen binding protein is from about 0.1 mg/kg to about 3.0 mg/kg.

37. A method according to claim 36, wherein the one or more symptoms is selected from the group consisting of: a reduction in fasting blood glucose; a reduction in glucose area under the curve (AUC) as determined by mixed meal tolerance test (MMTT); and a lowering of hemoglobin A1c (HbA1c) levels.

38. A method according to claim 37, wherein the isolated antagonistic antigen binding protein comprises an isolated antagonistic antibody and wherein the therapeutically effective amount of the isolated antagonistic antibody is selected from the group consisting of: about 0.1 to about 0.2 mg/kg, about 0.2 to about 0.4 mg/kg, about 0.4 to about 0.6 mg/kg, about 0.6 to about 0.8 mg/kg, about 0.8 to about 1.0 mg/kg, about 1.0 to about 1.2 mg/kg, about 1.2 to about 1.4 mg/kg, about 1.4 to about 1.6 mg/kg, about 1.6 to about 1.8 mg/kg, about 1.8 to about 2.0 mg/kg, about 2.0 to about 2.2 mg/kg, about 2.2 to about 2.4 mg/kg, about 2.4 to about 2.6 mg/kg, about 2.6 to about 2.8 mg/kg, and about 2.8 to about 3.0 mg/kg.

39. A method according to claim 20, wherein the isolated antagonistic antigen binding protein is admixed with a pharmaceutically acceptable carrier to form a pharmaceutical composition for systemic administration to the patient.
